(19) United States
(12) Patent Application Publication
(10) Pub. No.: US 2022/0020503 A1
(43) Pub. Date: Jan. 20, 2022

(54) PREDICTING EFFICACY OF PREVENTATIVE MEASURES TO MITIGATE SPREAD OF A PATHOGEN AND ILLNESSES CAUSED THEREFROM USING MACHINE LEARNING MODELS

(71) Applicant: General Genomics, Inc., Midland, TX (US)

(72) Inventors: Warren Dennis GIECK, Calgary (CA); Aronjol David ROSENTHAL, Midland, TX (US)

(21) Appl. No.: 17/207,485

(22) Filed: Mar. 19, 2021

Related U.S. Application Data
(60) Provisional application No. 63/053,941, filed on Jul. 20, 2020.

(51) Int. Cl. G16H 50/80; G06N 5/04; G06N 20/00; G06K 9/62

(52) U.S. Cl. CPC G16H 50/80; G06N 5/04; G06K 9/6256; G06K 9/6231; G06N 20/00

(57) ABSTRACT

Embodiments of the present disclosure generally relate to methods for analyzing the effectiveness of preventative measures on the spread of illnesses, such as COVID-19, on living organisms. More particularly, embodiments of the present disclosure relate to methods for identifying the effectiveness of preventative measures, processes, equipment and other available data, and providing indicators and methods of visualization the effectiveness of preventative measures on the spread of an illness.

### CROSS-REFERENCE TO RELATED APPLICATIONS

This application claims benefit to U.S. Provisional Patent Application Ser. No. 63/053,941, entitled “Using Data Science for the Determination of Safety Procedures and Equipment Effectiveness for the Prevention and Spread of COVID-19 and Other Pandemics in Businesses and Facilities,” filed Jul. 20, 2020 and assigned to the assignee hereof, the contents of which are hereby incorporated by reference in its entirety.

### BACKGROUND

### Field

Embodiments of the present disclosure generally relate to methods for analyzing effectiveness of preventative measures for the spread of COVID-19 and other pandemics in businesses and facilities.

### Description of the Related Art

Conventional methods for analyzing the preventative measures for the spread of COVID-19 and other pandemics are generally qualitative or completed in laboratory conditions and not quantitative or tested in real world scenarios.

Therefore, there is a need in the art for more accurate analysis of the preventative measures on the spread of COVID-19 and other pandemics.

### SUMMARY

Embodiments of the present disclosure generally relate to methods for analyzing effectiveness of preventative measures on the spread of illnesses, such as COVID-19. More particularly, embodiments of the present disclosure relate to methods for identifying the effectiveness of preventative measures using data collected from facilities that are using these measures and other available data.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope. The disclosure may admit to other equally effective embodiments.

FIG. 1 illustrates a flow chart of a method according to embodiments of the present disclosure.

FIG. 2 illustrates example operations that may be performed by a computing system to train one or more machine learning models to predict the efficacy of various preventative measures to mitigate the spread of a pathogen and illnesses caused therefrom, according to embodiments of the present disclosure.

FIG. 3 illustrates example operations that may be performed by a computing system to predict the efficacy of preventative measures to mitigate the spread of a pathogen and illnesses caused therefrom using one or more trained machine learning models, according to embodiments of the present disclosure.

FIG. 4 illustrates an example system in which embodiments of the present disclosure may be implemented.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Embodiments of the present disclosure generally relate to methods for analyzing the effectiveness of preventative measures on the spread of illnesses, such as COVID-19. More particularly, embodiments of the present disclosure relate to methods for identifying risk of illness based on preventative methods being used and other available data, and providing indicators and methods of visualization for effectiveness of preventative measures for facilities.

### Definitions

As used herein, “living organism” refers to any human, animal, plant or other organism that is living or was considered alive at any point.

As used herein, “facilities” refers to places of work, gathering, entertainment, worship, retail, sports venues, schools, restaurants, and more.

### DESCRIPTION

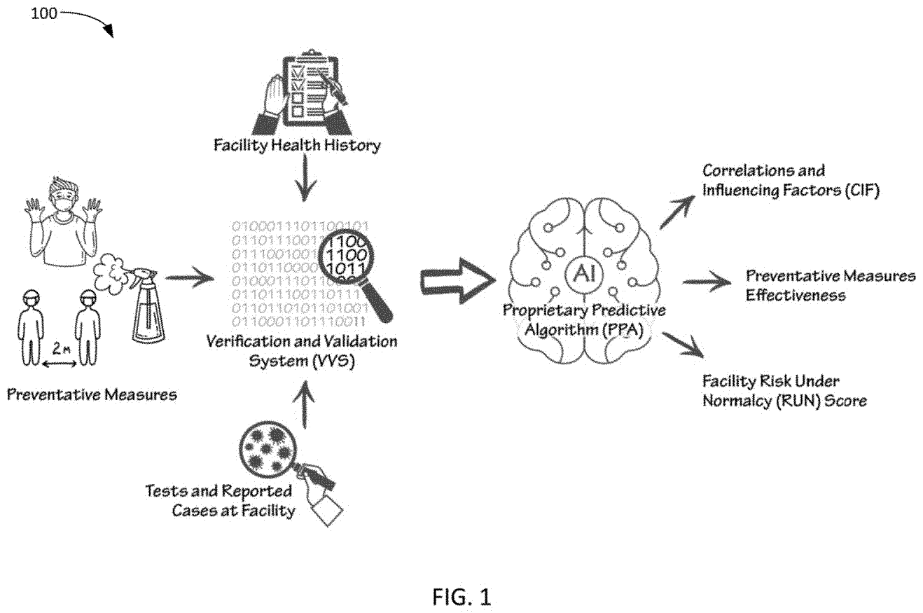

FIG. 1 illustrates a flow chart of a method 100 according to embodiments of the present disclosure. The method 100 generally includes collecting data, standardizing the collected data, generating a testing data set and a training data set, building a correlative model using machine learning, and providing predictions regarding effectiveness of preventative measures using the correlative model. As shown in FIG. 1, the method 100 includes generating quantitative predictions.

The minimum data required includes (1) classification of all forms of preventative measure(s) being used at a facility, such as sanitation methods, protective equipment used, air filtration, spacing/distancing, contactless entry, contactless payment, and others, (2) details of workers or patrons that have been identified with an illness, and (3) details of the number of individuals in the facility.

Other data includes, but is not limited to, all past, current, and future individual RUN scores or risk results, demographics (age, ethnicity, eye color, skin color, hair color, etc.), climate/location (location (ZIP/postal code)), date (current average weather, seasonality), environmental data (air quality, humidity, temperature), types of activities being held, and more.

At the conclusion of the method 100, certain quantitative output will be generated. Governmental or healthcare professionals, corporations, or other individuals may then use the quantitative output to undertake a risk assessment.

In certain embodiments, the method 100 may output certain risk data, such as a risk value regarding the percent of effectiveness for each preventative measure on the spread an illness, such as COVID-19. The risk value may then be reviewed and analyzed to determine whether the preventative measure is required or different measures should be put into place.

In certain embodiments, the collected data may include whether a preventative measure is effective for high risk individuals and/or whether a preventative measure is effective enough to allow those individuals to safely access and participate in the facility.

It is contemplated that the method 100 may be used to analyze large data sets, such as data from a hospital, to determine susceptibility of a population or subset thereof. It is also contemplated that the method 100 may be used as a self-assessment tool for a business or a facility to input data to determine their effectiveness in preventing the spread of an illness.

Embodiments of the present disclosure advantageously replace qualitative conjecture with quantitative evidence, utilizing data science to model the complex relationships as it pertains to illnesses. Embodiments of the present disclosure may be used by businesses and facilities to identify their own risks or by doctors, corporations or governments to prevent exposure for outbreaks that may affect any being.

### Example Prediction of Efficacy of Preventative Measures to Mitigate Spread of a Pathogen and Illnesses Caused Therefrom

From time to time, pathogens that may cause various illnesses in humans may arise. Some of these pathogens may be relatively common, such as the seasonal flu; other pathogens, such as the Ebola virus or novel coronaviruses (e.g., SARS-COV2, the virus that causes COVID-19) may be novel at the time they arise. Regardless of the novelty of a pathogen, to mitigate the spread of the pathogen and minimize the spread of illnesses caused by these pathogens, various actions can be implemented. For example, within a built environment, these actions may include surface sanitization, increases in spacing between people, the wearing of face coverings, introduction of various types of air filtration systems (e.g., positive pressure systems, faster air recycling systems, etc.), and/or other changes that may aid in mitigating the spread of a pathogen.

However, many of these actions may have an unknown, or at least an as-yet-quantified, effectiveness in mitigating the spread of a pathogen and illnesses caused therefrom. Laboratory testing of these various actions may, for example, indicate that a particular action reduces the amount of a pathogen in the air or on a surface by some amount. However, a reduction in the presence of a pathogen may not provide sufficient information to estimate the efficacy in mitigating the spread of a pathogen and illnesses caused by the spread of the pathogen in real-world environments.

Aspects of the present disclosure provide machine learning techniques that allow for the efficacy of various preventative measures to mitigate the spread of a pathogen (e.g., in a built environment) to be predicted, which in turn may be used to recommend actions to perform in order to mitigate the spread of the pathogen. By using machine learning models to predict the efficacy of various preventative measures in minimizing the spread of a pathogen based on real-world results from similar deployments, aspects of the present disclosure may allow for more accurate selection of preventative measures that will have the greatest effect in mitigating the spread of a pathogen. Preventative measures that are more likely to mitigate the spread of a pathogen may be identified and implemented over preventative measures that are less likely to cure or mitigate the effects of the medical condition. Thus, aspects of the present disclosure may promote reductions in illnesses caused by the pathogen.

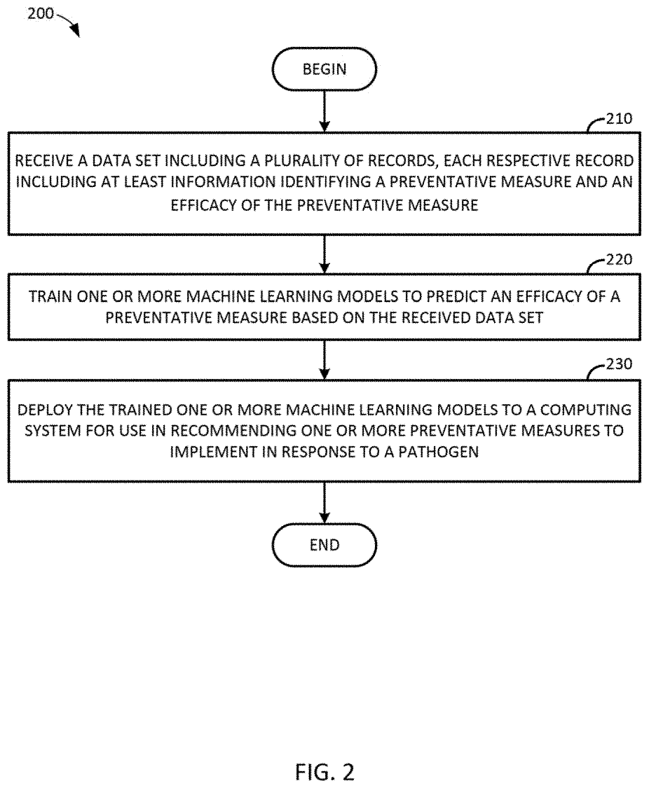

FIG. 2 illustrates example operations for training a machine learning model to predict the efficacy of preventative measures in mitigating the spread of a pathogen and illnesses caused therefrom, according to certain aspects described herein. The efficacy of these preventative measures may, in some aspects, be defined in terms of a number of cases of illnesses caused by the pathogen prior to and after implementation of the preventative measure(s) in a built environment. In some aspects, the efficacy of these measures may be related to attributes of the built environment in which these presentative measures are implemented, such as the size of the built environment.

As illustrated, operations 200 may begin at block 210, where a computing system receives a data set including a plurality of records. Each record in the plurality of records generally identifies at least a preventative measure and an efficacy of the preventative measure. Efficacy of the preventative measure may be represented, for example, as a reduction in persons having an illness caused by a pathogen relative to an estimated number of persons having the illness in the absence of the preventative measure or to a number of persons contracting the illness prior to implementation of the preventative measure.

In some aspects, efficacy of the preventative measure may be classified into one of a plurality of efficacy categories. For example, the efficacy of a preventative measure can be classified as ineffective (e.g., where the rate at which persons contract an illness does not materially change after implementation of the preventative measure); somewhat effective, or very effective. One or more rules may be used to map raw numbers or proportional changes to one of the plurality of categories of efficacy, and this category information can be used to train the one or more machine learning models to recommend preventative measures to implement in order to mitigate the spread of a pathogen and illnesses caused therefrom.

In some aspects, the data set may be received from a plurality of data sources and may be aggregated into a unified data set prior to training one or more machine learning models. The plurality of data sources may include, for example, a secure medical records repository (e.g., a repository of patient medical records subject to the privacy and security requirements of the Health Insurance Portability and Accountability Act or other relevant data privacy regulations) and from one or more other external data sources, such as activity trackers, patient surveys, exposure counters, wearable medical devices, or the like. Generally, to aggregate the data into the unified data set, patient information from each of a plurality of sources can be mapped to one or more attributes in the unified data set into which patient information is to be mapped, and the appropriate values may be filled into the attributes in the unified data set from the appropriate data source.

The attributes included in each record in the received data set may include a variety of environmental attributes associated with the environment in which the preventative measure was implemented, changes in the environment after implementing the preventative measure, or the like. Information about illnesses contracted from the pathogen prior to and after implementation of the preventative measure may be retrieved, for example, from individual patient records for patients associated with the environment and may be aggregated across individual patient records into a single value representing, for example, the number of persons who became ill after implementation of the preventative measure, a change in the numbers of persons who contracted an illness prior to and after implementation of the preventative measure, or the like.

In some aspects, the data set may be featurized prior to use in training one or more machine learning models to predict the efficacy of preventative measures in mitigating the spread of a pathogen and illnesses caused therefrom. To featurize the one or more attributes in each record, the raw data in each record in the received data set may be transformed into machine-readable or machine-usable data that can be used to train a machine learning model. Generally, raw data may be transformed into numerical data representing, for example, a binary choice (e.g., whether a preventative measure has had a downward impact on illnesses caused from a pathogen), one of a plurality of categories (e.g., where an attribute has a range of values, and different sub-ranges indicate, for example, relative sizes of a built environment in which the preventative measure is being implemented), or numerical data scaled based on a scaling factor.

The computing system can use one or more predefined rules to determine how to featurize one or more attributes in the data set. Each attribute to be included in a training data set may be associated with a rule indicating how the underlying raw data from the received data set is to be transformed into a feature usable in training a machine learning model to predict susceptibility to a medical condition. In some aspects, the rules may define how multiple related data items may be aggregated into a single value, and the single value may be featurized. In another example, multiple different values may map to a same featurized value, and the rule may define how different raw values map to a given featurized value. In another example, the rules may define upper and lower bound values for classification of an attribute into one of a plurality of categories.

In some aspects, some attributes may be determined based on raw data, and the one or more predefined rules may specify a scaling factor associated with the devices that recorded the raw data to use in scaling the data (e.g., prior to featurization). The scaling factor may be, for example, associated with an accuracy of a measurement device, which may be defined a priori according to manufacturer specifications or prior experience with the measurement device. For example, where an attribute includes a size of feature captured using one or more imaging devices, the raw size information may be adjusted based on an expected measurement error for the source imaging device. If, for example, an imaging device is known to be accurate to within n percent, the raw data may be scaled to a value of 100+n percent or 100−n percent, depending on the specific direction of error, developer choice, or the like. The scaled value may be preserved as the value associated with an attribute or may be further featurized into a binary feature or a feature with a fixed set of values, as discussed above.

In some aspects, the attributes included in the received data set may be reduced based on various filtering or selection techniques. It may be noticed, for example, that the records in the data set include similar values for a particular attribute, regardless of whether the preventative measure has greatly decreased the incidence of illnesses caused by a pathogen, moderately decreased the incidence of illnesses caused by the pathogen, or has had no impact on the incidence of illnesses caused by the pathogen. Because values for the particular attribute are similar for disparate outcomes across records in the data set, it may be determined that the attribute is not probative of whether a preventative measure is effective (or not effective) in reducing the spread of a pathogen and illnesses caused therefrom. Thus, the attribute may be removed from each of the records in the data set, which may reduce the amount of data processed while training the machine learning models. In another example, statistical tests can be used to determine whether an attribute is independent or dependent by using techniques such as chi-squared testing to determine whether observations deviate from an expected outcome for a particular analysis. In still further examples, various machine learning techniques can be used to assign an importance or significance value to each attribute. Attributes in the received data set having importance or significance values exceeding a threshold value may be retained in the received data set, while attributes having importance or significance values below the threshold value may be removed from the received data set.

In some aspects, the data set may not include a value for an attribute for a given record of a preventative measure. To allow for each of the records in the data set to have a same number of attributes, the record for that given preventative measure may be modified with a value for the attribute indicating that the attribute does not apply to the record associated with the preventative measure. For example, the value for the attribute may be a reserved value (e.g., a predefined magic number), a null value, or the like.

At block 220, the computing system trains one or more machine learning models to predict the efficacy of a preventative measure based on the received data set. The one or more machine learning models may be various types of machine learning models configured to generate various outputs. For example, the machine learning models may include one or more of probabilistic models, neural networks, clustering models, or other appropriate machine learning models. Generally, a probabilistic model may be configured to generate a probability distribution over a plurality of treatment options, where the probability value associated with a preventative measure corresponds to a likelihood that the preventative measure is effective in mitigating the spread of a pathogen and illnesses caused therefrom. A clustering algorithm may be used to identify records of preventative measures being implemented in similar environments. Information about the resulting efficacy from the similar environments can then be used, as discussed in further detail below, to identify recommended preventative measures. For example, recommended preventative measures may be identified based on an average amount of change in the number of persons contracting an illness associated with a pathogen after implementation of the preventative measure.

At block 230, the computing system deploys the trained one or more machine learning models to one or more other computing systems for use in recommending one or more preventative measures to implement in response to a pathogen.

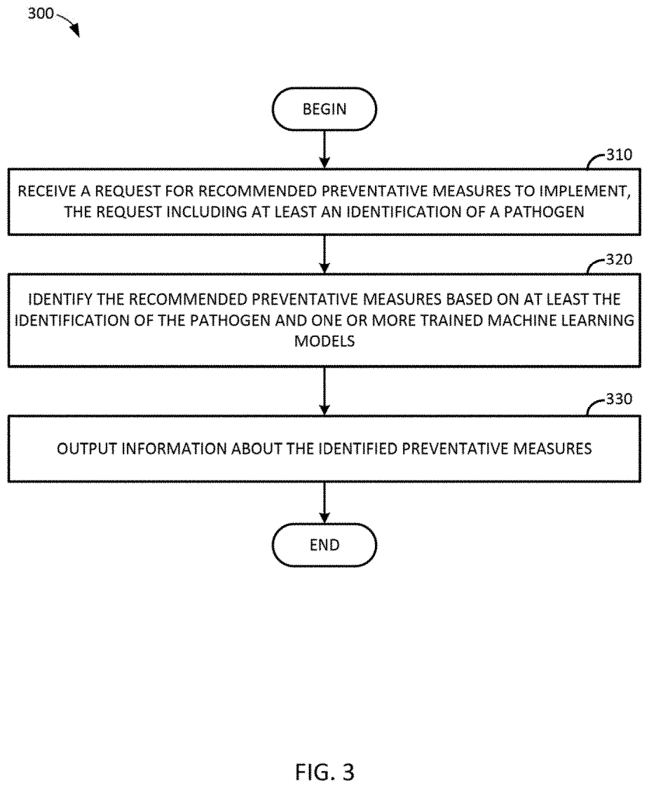

FIG. 3 illustrates example operations 300 that may be performed by a computing system to recommend preventative measures to implement (e.g., in a built environment) based on one or more machine learning models.

As illustrated, operations 300 may begin at block 310, where the computing system receives a request to identify one or more recommended preventative measures to implement. The request generally includes at least information identifying the pathogen against which preventative measures are to be taken. In some aspects, the request may include additional information, such as physical measurements of the built environment in which the preventative measure is to be implemented, an amount of traffic passing through the built environment over a given period of time, and other information that may influence the efficacy of a preventative measure. The information identifying the pathogen may include, for example, information about a general classification of the pathogen (e.g., whether the pathogen is a coronavirus, an ebolavirus, etc.), information about a mechanism through which the pathogen multiplies and spreads to other humans, a rate of spread, etc.

The attributes included in request may include a variety of medical, activity, environmental, and other information about the environment in which the preventative measure is to be implemented. In some aspects, the attributes may include information about the prevalence of various medical conditions of persons who are expected to be present in the built environment. This information may include, for example, information about a prevalence of various medical conditions that may impact susceptibility to a pathogen and symptomatic illnesses caused therefrom, such as a prevalence of chronic diseases, a prevalence of immune system disorders, a prevalence of obesity, and/or other conditions. Environmental information may include, for example, indications of various chemicals or types of radiation present in the built environment, the amount of exposure, and/or other environmental information that may influence susceptibility to an illness and impact the efficacy of preventative measures taken against a pathogen. In some aspects, the attributes included in the request may include information identifying preventative measures that have already been implemented; this information may be used to condition a machine learning model to generate recommendations based on a combination of the already-implemented preventative measures and one or more additional preventative measures.

At block 320, the computing system identifies one or more recommended preventative measures to implement by generating a prediction using one or more trained machine learning models. As discussed above, the machine learning models may have been previously trained based on a featurized data set associating, preventative measures with information about a pathogen to be controlled and efficacy of the preventative measures.

In some aspects, the one or more trained machine learning models may take a feature vector as input into the model and generate a prediction, such as a probability score or a cluster of similar records. To generate the feature vector, the computing system can transform the raw data in the request into machine-readable or machine-usable data that can be used to train a machine learning model. Generally, raw data may be transformed into numerical data representing, for example, a binary choice, one of a plurality of categories (e.g., where an attribute has a range of values, and different sub-ranges are probative of different levels of efficacy), or numerical data scaled based on a scaling factor.

The computing system can use one or more predefined rules to determine how to featurize each of the one or more attributes associated with a preventative measure. Each attribute to be used in predicting the efficacy of a preventative measure in mitigating the spread of a pathogen and illness caused therefrom may be associated with a rule indicating how the underlying raw data from the received data set is to be transformed into a feature usable by a machine learning model to predict the efficacy of preventative measures in mitigating the spread of a pathogen and illnesses caused therefrom. In some aspects, the rules may define how multiple related data items may be aggregated into a single value, and the single value may be featurized. In another example, multiple different values may map to a same featurized value. In another example, the rules may define upper and lower bound values for classification of an attribute into one of a plurality of categories.

In some aspects, some attributes may be determined based on raw data, and the one or more predefined rules may specify a scaling factor associated with the devices that recorded the raw data to use in scaling the data (e.g., prior to featurization). The scaling factor may be, for example, associated with an accuracy of a measurement device, which may be defined a priori according to manufacturer specifications or prior experience with the measurement device. The scaled value may be preserved as the value associated with an attribute or may be further featurized into a binary feature or a feature with a fixed set of values, as discussed above.

In some aspects, the attributes included in the request may be reduced based on various filtering or selection techniques. The filtering or selection techniques may be defined based on the filtering or selection techniques used to filter data in a training data set used to train the one or more machine learning models. To reduce the information included in the feature vector down to a minimal set of information needed for the one or more machine learning models to predict the efficacy of preventative measures in mitigating the spread of a pathogen and illnesses caused therefrom, attributes that are known a priori to not be probative of whether a preventative measure is effective in mitigating the spread of a pathogen and illnesses caused therefrom may be removed from the data set included in the request.

In some aspects, the data set may not include a value for an attribute. To allow for the feature vector to have a same number of attributes as the records in the training data set used to train the one or more machine learning models, the feature vector may be modified with a value for the attribute indicating that the attribute does not apply to the preventative measure. For example, the value for the attribute may be a reserved value (e.g., a predefined magic number), a null value, or the like.

In some aspects, the one or more machine learning models may include probabilistic models that are trained to output, for a given input, a probability distribution over a universe of possible outcomes. In some aspects, the probability distribution may be generated over each of the preventative measures for which data exists in a training data set, with the probability value associated with each preventative measure serving as a proxy for a likelihood of efficacy in mitigating the spread of a pathogen and illnesses caused therefrom. In some aspects, multiple probabilistic models can be used to predict which preventative measures are likely to be effective for the given environment in which the preventative measures will be installed, and each model of the multiple probabilistic models may be associated with a weighting value. A score serving as a proxy for the efficacy of preventative measures in mitigating the spread of a pathogen and illnesses caused therefrom may be calculated as a weighted average of the probability scores output by each of the multiple probabilistic models.

In some aspects, the one or more machine learning models may also or alternatively include one or more clustering models that are trained to identify a set of matching historical implementations of preventative measures having similar associated attributes. To identify recommended preventative measurements, a score can be generated based on the efficacy metrics associated with each preventative measure in the set of matching preventative measures. For example, a score may be generated based on a weighted average of the efficacy for each implementation of the preventative measure. In some aspects, a score for each treatment may also be adjusted based on the other parameters, such as a cost of implementing the preventative measure, an amount of time needed to implement the preventative measure, or the like. The scaling factor associated with these parameters may be used to scale the predicted efficacy of a preventative measure downwards to account delays or infeasibility in implementing the preventative measure. By doing so, the system can recommend preventative measures that are the most cost effective (e.g., recommending cheaper measures over more expensive measures, all else equal) or can be implemented more quickly.

In some aspects, a probabilistic model and a clustering model (as well as other machine learning models) may be used in conjunction with each other to predict the efficacy of preventative measures in mitigating the spread of a pathogen and illnesses caused therefrom. In one example, a probabilistic model may be associated with a first weighting value, and the clustering model may be associated with a second weighting value. The probability score—representing the efficacy of preventative measures in mitigating the spread of a pathogen and illnesses caused therefrom—may be calculated as sum of the score generated by the probabilistic model, weighted by the first weighting value, and the score generated by the clustering model, weighted by the second weighting value.

At block 330, the computing system outputs information about the identified preventative measures. The identified preventative measures may be, for example, an ordered list of preventative measures based on an efficacy score calculated for each preventative measure. Generally, higher scoring preventative measures (e.g., preventative measures with high predicted efficacy, low cost, and quick implementation) being at the top of the ordered list, and lower scoring treatments (e.g., preventative measures with low predicted efficacy or preventative measures with high predicted efficacy and high predicted cost or time to implement) being at the bottom of the ordered list.

### Example Systems for Recommending Preventative Measures to Mitigate the Spread of a Pathogen and Illnesses Caused Therefrom Using Machine Learning Models

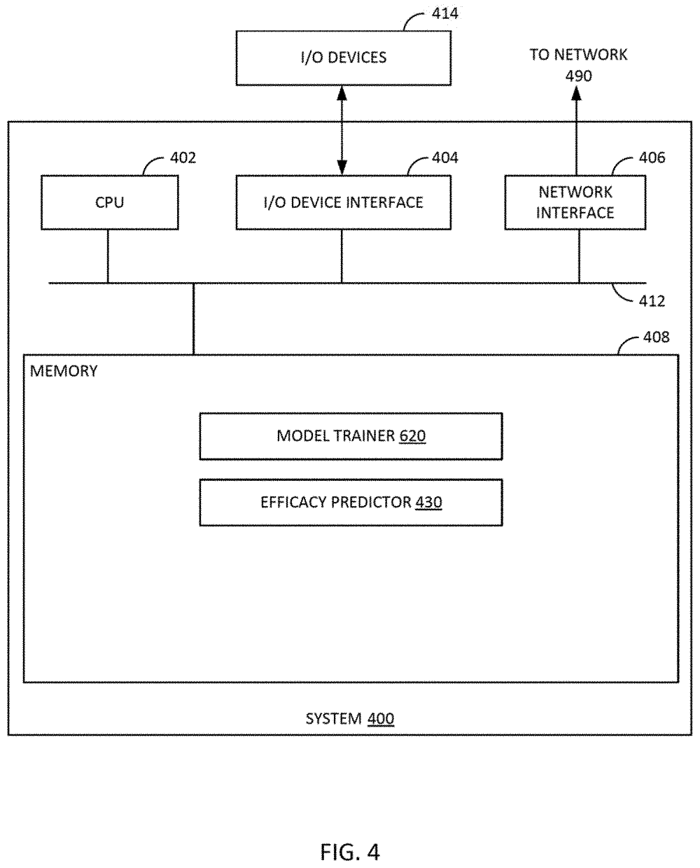

FIG. 4 illustrates an example system 400 that can train and use machine learning models to recommend preventative measures to implement (e.g., in a built environment) to mitigate the spread of a pathogen, according to certain embodiments described herein.

As shown, system 400 includes a central processing unit (CPU) 402, one or more I/O device interfaces 404 that may allow for the connection of various I/O devices 414 (e.g., keyboards, displays, mouse devices, pen input, etc.) to the system 400, network interface 406 through which system 400 is connected to network 460 (which may be a local network, an intranet, the internet, or any other group of computing devices communicatively connected to each other), a memory 408, storage 410, and an interconnect 412.

CPU 402 may retrieve and execute programming instructions stored in the memory 408. Similarly, the CPU 402 may retrieve and store application data residing in the memory 408. The interconnect 412 transmits programming instructions and application data, among the CPU 402, I/O device interface 404, network interface 404, memory 408, and storage 410.

CPU 402 is included to be representative of a single CPU, multiple CPUs, a single CPU having multiple processing cores, and the like.

Memory 408 is representative of a volatile memory, such as a random access memory, or a nonvolatile memory, such as nonvolatile random access memory, phase change random access memory, or the like. As shown, memory 608 includes a model trainer 420 and an efficacy predictor 430.

Model trainer 420 may be configured to perform the operations discussed herein (e.g., with respect to operations 200 illustrated in FIG. 2 and/or other operations) to train and deploy one or more machine learning models for recommending preventative measures to implement in order to mitigate the spread of a pathogen and illnesses caused therefrom. As discussed, model trainer 420 can receive data from a plurality of data sources (including, but not limited to, a secure medical records data source, a physical activity records data source, a medicine usage data source, and/or other data sources in which attributes that may be predictive, alone or in isolation, of efficacy of preventative measures may be stored) and generate a training data set by featurizing the one or more attributes. Model trainer 420 may be configured to train one or more machine learning models based on the generated training data set. As discussed, the one or more machine learning models may include probabilistic models, clustering-based models, and/or other machine learning models that may be used to recommend preventative measures to implement to mitigate spread of a pathogen and illnesses caused therefrom. Model trainer 420 may then deploy the trained one or more machine learning models for use (e.g., to efficacy predictor 430 and/or one or more external computing systems accessible via network 460).

Efficacy predictor 430 may be configured to perform the operations discussed herein (e.g., with respect to operations 300 illustrated in FIG. 3 and/or other operations) to identify preventative measures to implement in order to mitigate the spread of a pathogen and illnesses caused therefrom using one or more machine learning models. As discussed, efficacy predictor 430 may use the one or more machine learning models trained by model trainer 420 to predict the efficacy of preventative measures in mitigating the spread of a pathogen and illnesses caused therefrom and recommend preventative measures to implement (e.g., in a built environment). To do so, efficacy predicator 430 can receive a request including a data set of attributes about an environment in which a preventative measure is to be implemented and the pathogen against which the preventative measure is targeted and generate a feature vector based on the data set of attributes associated with a preventative measure. The feature vector may be provided as input into one or more machine learning models to generate a score for each of a plurality of preventative measures. Based on the generated scores, efficacy predictor 430 can identify one or more preventative measures that are candidates for implementation in the environment specified in the request. These preventative measures may be, for example, preventative measures having high efficacy, lower cost, and quicker implementation.

### Additional Considerations

The preceding description is provided to enable any person skilled in the art to practice the various embodiments described herein. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. Also, features described with respect to some examples may be combined in some other examples. For example, an apparatus may be implemented or a method may be practiced using any number of the embodiments set forth herein. In addition, the scope of the disclosure is intended to cover such an apparatus or method that is practiced using other structure, functionality, or structure and functionality in addition to, or other than, the various embodiments of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

A processing system may be implemented with a bus architecture. The bus may include any number of interconnecting buses and bridges depending on the specific application of the processing system and the overall design constraints. The bus may link together various circuits including a processor, machine-readable media, and input/output devices, among others. A user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer-readable medium. Software shall be construed broadly to mean instructions, data, or any combination thereof, whether referred to as software, firmware, middleware, microcode, hardware description language, or otherwise. Computer-readable media include both computer storage media and communication media, such as any medium that facilitates transfer of a computer program from one place to another. The processor may be responsible for managing the bus and general processing, including the execution of software modules stored on the computer-readable storage media. A computer-readable storage medium may be coupled to a processor such that the processor can read information from, and write information to, the storage medium. In the alternative, the storage medium may be integral to the processor. By way of example, the computer-readable media may include a transmission line, a carrier wave modulated by data, and/or a computer readable storage medium with instructions stored thereon separate from the wireless node, all of which may be accessed by the processor through the bus interface. Alternatively, or in addition, the computer-readable media, or any portion thereof, may be integrated into the processor, such as the case may be with cache and/or general register files. Examples of machine-readable storage media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module, it will be understood that such functionality is implemented by the processor when executing instructions from that software module.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various embodiments described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method for predicting efficacy of preventative measures to mitigate spread of a pathogen and illnesses caused therefrom, comprising:
receiving a data set including a plurality of records, each respective record including at least information identifying a preventative measure and an efficacy of the preventative measure;
training one or more machine learning models to predict an efficacy of a preventative measure based on the received data set; and
deploying the trained one or more machine learning models to a computing system for use in recommending one or more preventative measures to implement in response to a pathogen.

2. The method of claim 1, further comprising:
generating a training data set by featurizing the received data set by assigning, for each respective attribute in the data set, one of a plurality of values, each value indicating a classification of the respective attribute into one of a plurality of categories,
wherein the one or more machine learning models are trained using the generated training data set.

3. The method of claim 1, further comprising:
adjusting values associated with an attribute in the data set based on a scaling factor associated with an accuracy of a source from which the value was obtained,
wherein the one or more machine learning models are trained based on the data set with the adjusted values.

4. The method of claim 1, further comprising:
replacing null values for attributes in the data set with an indication that the attributes do not apply to the preventative measure.

5. The method of claim 1, wherein the efficacy of the preventative measure comprises a reduction in persons contracting an illness caused by the pathogen relative to an estimated number of persons contracting the illness if no preventative measures were taken.

6. The method of claim 1, wherein records in the data set are aggregated from data retrieved from a plurality of external data sources.

7. The method of claim 6, wherein the plurality of external data sources comprises a secure medical records data source and one or more other data sources.

8. The method of claim 7, wherein the one or more other data sources include one or more of a physical activity records data source, or a patient medicine usage data source.

9. The method of claim 1, wherein the one or more machine learning models comprise clustering-based machine learning models.

10. The method of claim 1, wherein the one or more machine learning models comprise probabilistic models in which efficacy of the preventative measure is represented by a probability distribution over a plurality of preventative measures associated with similar pathogens.

11. A method for recommending preventative measures to implement to mitigate spread of a pathogen and illnesses caused therefrom, comprising:
receiving a request for recommended preventative measures to implement, the request including at least an identification of the pathogen;
identifying the recommended preventative measures based on at least the identification of the pathogen and one or more trained machine learning models; and
outputting information about the identified preventative measures.

12. The method of claim 11, wherein the one or more trained machine learning models comprise one or more probabilistic models trained to generate a probability distribution over a plurality of efficacy categories.

13. The method of claim 12, wherein identifying the preventative measures comprises generating a probability score as a weighted average of efficacy probabilities generated by each of the one or more trained machine learning models, each model of the one or more trained learning model being associated with a weighting value to assign to a predicted efficacy of the preventative measures.

14. The method of claim 11, wherein the one or more trained machine learning models comprise one or more clustering models trained to identify a set of preventative measures undertaken in response to at least the identified pathogen.

15. The method of claim 14, wherein identifying the recommended preventative measures comprises:
grouping the identified set of preventative measures into a plurality of sub-groups, each sub-group being associated with a specific preventative measure in the identified set;
for each sub-group, calculating an average efficacy of the associated specific preventative measure; and
selecting, from the identified set of preventative measures, one or more measures having a calculated average efficacy above a threshold value.

16. The method of claim 11, wherein:
the one or more trained machine learning models comprise a probabilistic model configured to output a probability distribution over a plurality of efficacy categories and a clustering model configured to identify a set of preventative measures undertaken in response to at least the identified pathogen, and
the identifying the recommended preventative measures is based on a weighted average of probability scores in the probability distribution and average efficacy of preventative measures in the identified set of preventative measures.

17. The method of claim 11, wherein the received request includes information identifying preventative measures that have already been implemented.

18. The method of claim 17, wherein identifying the recommended preventative measures comprises selecting one or more other preventative measures having a predicted efficacy exceeding a predicted efficacy associated with the preventative measures that have already been implemented.

19. The method of claim 11, wherein the received request includes information about a physical environment in preventative measures are to be implemented, and the identifying the recommended preventative measures is further based on the information about the physical environment.

20. A system for recommending preventative measures to implement to mitigate spread of a pathogen and illnesses caused therefrom, comprising:
a memory having instructions stored thereon; and
a processor configured to execute the instructions to cause the system to:
receive a request for recommended preventative measures to implement, the request including at least an identification of the pathogen;
identify the recommended preventative measures based on at least the identification of the pathogen and one or more trained machine learning models; and
output information about the identified preventative measures.

21. A method for predicting efficacy of preventative measures to mitigate spread of a pathogen and illnesses caused therefrom, comprising:
receiving a data set including a plurality of records, each respective record including at least information identifying a preventative measure, an efficacy of the preventative measure, a pathogen against which the preventative measure is targeted, and information about a built environment in which the preventative measure is installed;
training one or more machine learning models to predict an efficacy of a preventative measure based on the received data set; and
deploying the trained one or more machine learning models to a computing system for use in recommending one or more preventative measures to implement in response to a pathogen.

22. The method of claim 21, further comprising:
generating a training data set by featurizing the received data set by assigning, for each respective attribute in the data set, one of a plurality of values, each value indicating a classification of the respective attribute into one of a plurality of categories,
wherein the one or more machine learning models are trained using the generated training data set.

23. The method of claim 21, further comprising:
adjusting values associated with an attribute in the data set based on a scaling factor associated with an accuracy of a source from which the value was obtained,
wherein the one or more machine learning models are trained based on the data set with the adjusted values.

24. The method of claim 21, further comprising:
replacing null values for attributes in the data set with an indication that the attributes do not apply to the preventative measure.

25. The method of claim 21, wherein the efficacy of the preventative measure comprises a reduction in persons contracting an illness caused by the pathogen over a time window after implementation of the preventative measure relative to a number of persons contracting the illness in the time window prior to implementation of the preventative measure.

26. The method of claim 21, wherein records in the data set are aggregated from data retrieved from a plurality of external data sources.

27. The method of claim 26, wherein the plurality of external data sources comprises a secure medical records data source and one or more other data sources.

28. The method of claim 27, wherein the one or more other data sources include one or more of a physical activity records data source, or a patient medicine usage data source.

29. The method of claim 21, wherein the one or more machine learning models comprise clustering-based machine learning models.

30. The method of claim 21, wherein the one or more machine learning models comprise probabilistic models in which efficacy of the preventative measure is represented by a probability distribution over a plurality of preventative measures associated with similar pathogens.

31. A method for recommending preventative measures to implement to mitigate spread of a pathogen and illnesses caused therefrom, comprising:
receiving a request for recommended preventative measures to implement, the request including at least an identification of the pathogen and information about a built environment in which a preventative measure is to be implemented;
identifying the recommended preventative measures based on at least the identification of the pathogen and one or more trained machine learning models; and
outputting information about the identified preventative measures.

32. The method of claim 21, wherein the one or more trained machine learning models comprise one or more probabilistic models trained to generate a probability distribution over a plurality of efficacy categories.

33. The method of claim 32, wherein identifying the preventative measures comprises generating a probability score as a weighted average of efficacy probabilities generated by each of the one or more trained machine learning models, each model of the one or more trained learning model being associated with a weighting value to assign to a predicted efficacy of the preventative measures.

34. The method of claim 31, wherein the one or more trained machine learning models comprise one or more clustering models trained to identify a set of preventative measures undertaken in response to at least the identified pathogen.

35. The method of claim 34, wherein identifying the recommended preventative measures comprises:
grouping the identified set of preventative measures into a plurality of sub-groups, each sub-group being associated with a specific preventative measure in the identified set;
for each sub-group, calculating an average efficacy of the associated specific preventative measure; and
selecting, from the identified set of preventative measures, one or more measures having a calculated average efficacy above a threshold value.

36. The method of claim 31, wherein:
the one or more trained machine learning models comprise a probabilistic model configured to output a probability distribution over a plurality of efficacy categories and a clustering model configured to identify a set of preventative measures undertaken in response to at least the identified pathogen, and
the identifying the recommended preventative measures is based on a weighted average of probability scores in the probability distribution and average efficacy of preventative measures in the identified set of preventative measures.

37. The method of claim 31, wherein the received request includes information identifying preventative measures that have already been implemented.

38. The method of claim 37, wherein identifying the recommended preventative measures comprises selecting one or more other preventative measures having a predicted efficacy exceeding a predicted efficacy associated with the preventative measures that have already been implemented.

39. The method of claim 31, wherein the received request includes information about a physical environment in preventative measures are to be implemented, and the identifying the recommended preventative measures is further based on the information about the physical environment.

40. A system for recommending preventative measures to implement to mitigate spread of a pathogen and illnesses caused therefrom, comprising:
a memory having instructions stored thereon; and
a processor configured to execute the instructions to cause the system to:
receive a request for recommended preventative measures to implement, the request including at least an identification of the pathogen;
identify the recommended preventative measures based on at least the identification of the pathogen and one or more trained machine learning models; and
output information about the identified preventative measures.
